(19) United States
(12) Patent Application Publication
(10) Pub. No.: US 2026/0165974 A1
(43) Pub. Date: Jun. 18, 2026

(54) Moisture-proof L-a-glycerylphosphorylcholine

(71) Applicant: NANJING NUTRABUILDING BIO-TECH CO., LTD., Nanjing (CN)

(72) Inventors: Jun TANG, Nanjing (CN); Wei WANG, Nanjing (CN); Chengfei WANG, Nanjing (CN); Xi ZHU, Nanjing (CN); Kylin LIAO, Nanjing (CN); Shawn WELLS, Frisco, TX (US)

(21) Appl. No.: 19/536,812

(22) Filed: Feb. 11, 2026

Related U.S. Application Data
(63) Continuation of application No. PCT/CN2024/112052, filed on Aug. 14, 2024.

(30) Foreign Application Priority Data
Aug. 15, 2023 (WO) PCT/CN2023/113060

(51) Int. Cl. A61K 9/20; A61K 9/16; A61K 47/10; A61K 47/14; A61K 47/18

(52) U.S. Cl. CPC A61K 9/2054; A61K 9/1635; A61K 9/2018; A61K 9/2027; A61K 9/2077; A61K 47/10; A61K 47/14; A61K 47/183

(57) ABSTRACT

The invention provides a moisture-proof L-α-glycerylphosphorylcholine and a preparation method thereof. Through the auxiliary material selection and preparation method treatment of raw materials, the problems of strong moisture absorption and deliquescence of L-α-glycerylphosphorylcholine when exposed to air are solved, and the excellent moisture-proof and deliquescence-resistant effects are achieved, which improves the stability of L-α-glycerylphosphorylcholine and brings high applicability to users.

### CROSS REFERENCE TO RELATED APPLICATION

This application is a continuation-in-part application of International Patent Application No. PCT/CN2024/112052, filed on Aug. 14, 2024, which claims the priority of the International Application No. PCT/CN2023/113060, filed on Aug. 15, 2023, the contents of all of which are incorporated herein by reference in their entirety.

### FIELD OF THE INVENTION

The invention belongs to the technical field of dietary supplements and/or preparations; and particularly relates to a moisture-proof L-α-glycerylphosphorylcholine and a preparation method thereof.

### BACKGROUND OF THE INVENTION

L-α-glycerylphosphorylcholine (GPC) is also known as glycerophosphorylcholine and choline glycerophosphate. It is the product that two fatty acyl groups on phosphatidylcholine (that is, lecithin) molecule are completely hydrolyzed, and it is a water-soluble phospholipid precursor and neurotransmitter naturally existing in human body. GPC is a unique cytoprotective agent that can support the fluidity and integrity of cell membrane. Years of experimental research and clinical application show that GPC can not only improve people's memory and cognitive ability, but also have a significant effect on cerebral circulation decline and senile dementia diseases such as Alzheimer's disease, so it is called anti-aging nutrient for the brain. In addition, it can also resist muscle atrophy and protect blood vessels. Therefore, GPC has important research value in the field of dietary supplements and health care.

The molecular formula of L-α-glycerylphosphorylcholine is C₈H₂₀NO₆P, and its relative molecular weight is 257.2. GPC molecule is a kind of “dehydrohexyl” phosphatidylcholine without fatty acid tail, which has both positive and negative charges, and is hydrophilic and lipophilic. GPC is very hygroscopic, soluble in water, methanol and ethanol, and insoluble in other organic solvents. L-α-glycerylphosphorylcholine belongs to choline substances. It has strong moisture absorption and deliquescence when exposed to air, and its stability is very poor. When it is used, it will often cause moisture absorption and agglomeration, which will bring a lot of inconvenience. Therefore, it is urgent to develop a product of moisture-proof L-α-glycerylphosphorylcholine to solve the problems such as moisture absorption when exposed to air, and this product will have a very broad market prospect.

### SUMMARY OF THE INVENTION

In order to solve the above problems, the present invention provides a moisture-proof L-α-glycerylphosphorylcholine and a preparation method thereof. Through the auxiliary material selection and preparation method treatment of raw materials, the problems of strong moisture absorption and deliquescence of L-α-glycerylphosphorylcholine when exposed to air are solved, and the excellent moisture-proof and deliquescence-resistant effects are achieved, which improves the stability of L-α-glycerylphosphorylcholine, brings high applicability to users, and achieves high content that the existing moisture-proof L-α-glycerylphosphorylcholine products on the market cannot achieve.

In one aspect, the invention provides a moisture-proof L-α-glycerylphosphorylcholine, wherein the moisture-proof L-α-glycerylphosphorylcholine comprises, by weight, not less than 50% of L-α-glycerylphosphorylcholine.

In some embodiments, the moisture-proof L-α-glycerylphosphorylcholine comprises, by weight, not less than 10%, 15%, 20%, 25%, 30%, 35%, 40%, 45%, 50% of L-α-glycerylphosphorylcholine. In some embodiments, the moisture-proof L-α-glycerylphosphorylcholine comprises, by weight, not less than 51%, 52%, 53%, 54%, 55%, 56%, 57%, 58%, 59%, 60%, 61%, 62%, 63%, 64%, 65%, 70%, 75%, 80%, 85%, 90%, 95%, 97%, 99% of L-α-glycerylphosphorylcholine. In some embodiments, the moisture-proof L-α-glycerylphosphorylcholine comprises, by weight, 10-99%, 15-98%, 20-97%, 25-96%, 30-95%, 35-94%, 40-93%, 45-92%, 50-90% of L-α-glycerylphosphorylcholine. In some embodiments, the moisture-proof L-α-glycerylphosphorylcholine comprises, by weight, 50-99%, 51-98%, 52-97%, 55-96%, 58-95%, 60-95%, 65-92%, 70-90% of L-α-glycerylphosphorylcholine.

In some embodiments, the moisture-proof L-α-glycerylphosphorylcholine comprises, by weight, 51-99% of L-α-glycerylphosphorylcholine; 1-49% of one or more auxiliary materials selected from silicon dioxide, maltodextrin, mannitol, silica gel.

In some embodiments, the moisture-proof L-α-glycerylphosphorylcholine further comprises, by weight, 1-9% of one or more auxiliary materials selected from hydroxypropyl methylcellulose, lecithin or anhydrous sodium citrate.

In some embodiments, the moisture-proof L-α-glycerylphosphorylcholine comprises, by weight, 51-91% of L-α-glycerylphosphorylcholine; 1-41% of one or more of silicon dioxide, maltodextrin, mannitol; 1-8% of one or more of hydroxypropyl methylcellulose, lecithin or anhydrous sodium citrate.

In some embodiments, the moisture-proof L-α-glycerylphosphorylcholine comprises, by weight, 50-90% of L-α-glycerylphosphorylcholine; 1-5% of one or more auxiliary materials selected from silicon dioxide, magnesium stearate, calcium hydrogen phosphate, calcium silicate, micro-powder silica gel; 5-40% of one or more auxiliary materials selected from microcrystalline cellulose, compressible starch, lactose or mannitol; and 1-9% of one or more auxiliary materials selected from hydroxypropyl methylcellulose, sodium alginate, Arabic gum, guar gum.

In some embodiments, the moisture-proof L-α-glycerylphosphorylcholine comprises, by weight, 50-90% of L-α-glycerylphosphorylcholine; 1-5% of silicon dioxide; 5-40% of microcrystalline cellulose; 1-9% of hydroxypropyl methylcellulose.

In some embodiments, the moisture-proof L-α-glycerylphosphorylcholine comprises, by weight, 50-80% of L-α-glycerylphosphorylcholine; 1-5% of silicon dioxide, 10-40% of microcrystalline cellulose, 4-9% of hydroxypropyl methylcellulose.

In some embodiments, the moisture-proof L-α-glycerylphosphorylcholine is in granular or powdery form.

In some embodiments, the particle size of the moisture-proof L-α-glycerylphosphorylcholine is 10-100 meshes. In some embodiments, the particle size of the moisture-proof L-α-glycerylphosphorylcholine is 20-80 meshes.

In some embodiments, the moisture absorption weight gain of the moisture-proof L-α-glycerylphosphorylcholine is not more than 5.0%, 5.5%, 6.0%, 6.5% after being placed under high humidity conditions, e.g., at temperature of 25° C. and relative humidity of 75% for 120 min. In some embodiments, the moisture absorption weight gain of the moisture-proof L-α-glycerylphosphorylcholine is not more than 5.0%, 5.5%, 6.0%, 6.3%, 6.5%, 6.8%, 7.0%, 7.2% after being placed for a period of time. In some embodiments, the moisture absorption weight gain of the moisture-proof L-α-glycerylphosphorylcholine is not more than 6.3%, 6.5%, 6.8%, 7.0%, 7.2% after being placed under high humidity conditions, e.g., at temperature of 25° C. and relative humidity of 75% for 180 min. In some embodiments, the moisture absorption weight gain of the moisture-proof L-α-glycerylphosphorylcholine is not more than 4.2%, 4.5% after being placed under high humidity conditions, e.g., at temperature of 25° C. and relative humidity of 75% for 60 min.

In some embodiments, the repose angle of the moisture-proof L-α-glycerylphosphorylcholine is sustainably unchanged or changes not more than 8°, 10°, 12° after being placed for a period of time. In some embodiments, the repose angle of the moisture-proof L-α-glycerylphosphorylcholine is not higher than 44°, 45°, 48°, 50° after being placed under high humidity conditions, e.g., at temperature of 25° C. and relative humidity of 75% for 3 h.

In some embodiments, the passing rate of 20-mesh sieve of the moisture-proof L-α-glycerylphosphorylcholine is not less than 75%, 80%, 85%, 90%, 92% after being placed under high humidity conditions, e.g., at temperature of 25° C. and relative humidity of 75% for 20 min. In some embodiments, the passing rate of 20-mesh sieve of the moisture-proof L-α-glycerylphosphorylcholine is not less than 50%, 60%, 65%, 70%, 72% after being placed under high humidity conditions, e.g., at temperature of 25° C. and relative humidity of 75% for 60 min. In some embodiments, the passing rate of 20-mesh sieve of the moisture-proof L-α-glycerylphosphorylcholine is not less than 40%, 50%, 60% after being placed under high humidity conditions, e.g., at temperature of 25° C. and relative humidity of 75% for 180 min. In some embodiments, under high humidity conditions, e.g., at temperature of 25° C. and relative humidity of 75%, the passing rate of 20-mesh sieve of the moisture-proof L-α-glycerylphosphorylcholine is not less than 90%, 95%, 96% after 10 min; not less than 70%, 75%, 80%, 82% after 30 min; not less than 50%, 60%, 63% after 120 min.

In some embodiments, the moisture-proof L-α-glycerylphosphorylcholine is formulated into nutritional supplement, food, beverage or animal feed.

In some embodiments, the moisture-proof L-α-glycerylphosphorylcholine is prepared into solid preparation or liquid preparation.

In another aspect, the invention provides a method for preparing moisture-proof L-α-glycerylphosphorylcholine, which comprises the following steps: preparing hydroxypropyl methylcellulose of a prescribed amount into an ethanol solution; spraying on L-α-glycerylphosphorylcholine and one or more auxiliary materials selected from silicon dioxide, maltodextrin, mannitol, silica gel of prescribed amounts to obtain an auxiliary material-sprayed material; sieving to obtain the moisture-proof L-α-glycerylphosphorylcholine.

In some embodiments, the moisture-proof L-α-glycerylphosphorylcholine comprises, by weight, 51-99% of L-α-glycerylphosphorylcholine; 1-49% of one or more auxiliary materials selected from silicon dioxide, maltodextrin, mannitol, silica gel.

In some embodiments, the method further comprises preparing one or more auxiliary materials of a prescribed amount selected from lecithin or anhydrous sodium citrate into an ethanol solution before sieving, and spraying on the auxiliary material-sprayed material.

In some embodiments, the moisture-proof L-α-glycerylphosphorylcholine comprises, by weight, 51-91% of L-α-glycerylphosphorylcholine; 1-41% of one or more of silicon dioxide, maltodextrin, mannitol; 1-8% of one or more of hydroxypropyl methylcellulose, lecithin or anhydrous sodium citrate.

In some embodiments, the spraying is one of top spraying, side spraying or bottom spraying.

In another aspect, the invention provides a method for preparing moisture-proof L-α-glycerylphosphorylcholine, which comprises the following steps: mixing the following components according to formula: L-α-glycerylphosphorylcholine; one or more auxiliary materials selected from silicon dioxide, magnesium stearate, calcium hydrogen phosphate, calcium silicate, micro-powder silica gel; one or more auxiliary materials selected from microcrystalline cellulose, compressible starch, lactose or mannitol; one or more auxiliary materials selected from hydroxypropyl methylcellulose, sodium alginate, Arabic gum, guar gum; granulating after mixing; sieving to obtain the moisture-proof L-α-glycerylphosphorylcholine.

In some embodiments, the moisture-proof L-α-glycerylphosphorylcholine comprises, by weight, 50-90% of L-α-glycerylphosphorylcholine; 1-5% of silicon dioxide; 5-40% of microcrystalline cellulose; 1-9% of hydroxypropyl methylcellulose.

In some embodiments, the granulation is one of wet granulation, fluidized bed granulation, spray granulation, melt granulation, dry granulation.

In some embodiments, the granulation is dry granulation.

In some embodiments, the moisture-proof L-α-glycerylphosphorylcholine prepared by the method is used for preparing food, beverage, nutrition, skin care products or cosmetics.

This summary is provided to introduce the selection of concepts in a simplified form, which are further described in the detailed description below. Summary is not intended to identify key features or essential features of the claimed subject matter, nor is it intended to be used to limit the scope of the claimed subject matter.

### DETAILED DESCRIPTION OF THE INVENTION

Reference will now be made in detail to the preferred embodiments of the present disclosure, examples of which are further illustrated. While the present disclosure will be described in conjunction with the preferred embodiments, it will be understood that they are not intended to limit the present disclosure to these embodiments. To the contrary, the present disclosure is intended to cover alternatives, modifications and equivalents, which may be included within the spirit and scope of the present disclosure as defined by the claims.

As used herein, the term “or” is meant to include both “and” and “or.” In other words, the term “or” may also be replaced with “and/or.”

As used herein, the singular forms “a,” “an” and “the” are intended to include the plural forms as well, unless the context clearly indicates otherwise.

As used herein, the term “comprise” or “include” or their conjugations, refer to a situation where said terms are used in their non-limiting sense to mean that items following the word are included, but items not specifically mentioned are not excluded. It also encompasses the more limiting verb ‘to consist essentially of’ and ‘to consist of’.

### EXAMPLES

In the present disclosure, unless otherwise specified, scientific and technical terms used herein have the meanings commonly understood by those skilled in the art. Moreover, the laboratory operation steps used herein are all routine steps widely used in the corresponding fields.

The raw materials or reagents used in the present disclosure are commercially available unless otherwise specified. All reagents are commercial grade and used according to the received standards.

In one aspect, the invention provides a moisture-proof L-α-glycerylphosphorylcholine. The moisture-proof L-α-glycerylphosphorylcholine of the invention has excellent moisture-proof and deliquescence-resistant effects, improves the stability of L-α-glycerylphosphorylcholine, and brings extremely high applicability to users. The L-α-glycerylphosphorylcholine used in the present invention can be prepared and obtained by known methods.

In another aspect, the invention provides a method for preparing moisture-proof L-α-glycerylphosphorylcholine. In the invention, the GPC raw materials are screened for granulation mode, and granulation can be carried out in a fluidized bed mode, and the fluidized bed granulation, spraying can adopt three granulation modes of top spraying, side spraying and bottom spraying, and top spraying granulation is preferred. According to the invention, L-α-glycerylphosphorylcholine is granulated in a fluidized bed mode aiming at the selected auxiliary materials, so that mixing, granulating and drying can be completed at one time, the production process is simple, the degree of automation is high, and the obtained product is round and uniform, and the solubility is good. In this method, one or more auxiliary materials selected from microcrystalline cellulose, compressible starch, lactose or mannitol, and one or more auxiliary materials selected from hydroxypropyl methylcellulose, sodium alginate, Arabic gum, guar gum, lecithin or anhydrous sodium citrate can be prepared into ethanol solutions with a solid content of 5% respectively, and then sprayed onto L-α-glycerylphosphorylcholine. A 10-100 mesh, preferably 20-80 mesh sieve can be used to sieve, to obtain the moisture-proof L-α-glycerylphosphorylcholine. In this method, mannitol has good anti-moisture absorption and anti-caking and anti-sticking effects; coating materials such as hydroxypropyl methylcellulose can resist moisture absorption by blocking moisture.

In addition, by screening the granulation methods of GPC raw materials, the granulation methods such as wet granulation, fluidized bed granulation, spray granulation, melt granulation and dry granulation can be screened, and the dry granulation method is preferred. The adopted dry granulation process is suitable for the formula of the invention, which does not cause bed collapse, has low process energy consumption, low production cost, no solvent intervention in the preparation process, green production process and no waste gas and waste water. The addition of microcrystalline cellulose and the like can achieve good anti-caking effect in this formula, the use of hydroxypropyl methylcellulose and the like contributes to the further preparation application of moisture-proof L-α-glycerylphosphorylcholine, and silicon oxide and the like have good anti-caking and anti-sticking effects. The combination of these three materials has the advantages of high production yield, remarkable anti-moisture effect and economic and easily available auxiliary materials. In this method, a 10-100 mesh, preferably 20-80 mesh sieve can be selected to sieve, to obtain the moisture-proof L-α-glycerylphosphorylcholine.

The method realizes the preparation of high-content moisture-proof L-α-glycerylphosphorylcholine by screening suitable auxiliary materials and granulation methods, and the prepared moisture-proof L-α-glycerylphosphorylcholine achieves excellent moisture-proof and deliquescence-resistant effects, improves the stability of L-α-glycerylphosphorylcholine, and brings high applicability to users.

The following examples are illustrative of selected embodiments of the present disclosure and are not meant to limit the scope of the present disclosure.

### Example 1 Preparation of Moisture-Proof L-α-Glycerylphosphorylcholine

A moisture-proof L-α-glycerylphosphorylcholine comprises the following components in percentage by weight:

| | |
|---|---|
| L-α-glycerylphosphorylcholine | 51% |
| silicon dioxide | 1% |
| maltodextrin | 40% |
| hydroxypropyl methylcellulose | 8%. |

The preparation method was as follows: hydroxypropyl methylcellulose of a prescribed amount is prepared into an ethanol solution with a solid content of 5%. It was then sprayed on L-α-glycerylphosphorylcholine, maltodextrin and silicon dioxide of prescribed amounts. After granulation, it was sieved with a 40-mesh sieve to obtain the moisture-proof L-α-glycerylphosphorylcholine.

### Example 2 Preparation of Moisture-Proof L-α-Glycerylphosphorylcholine

A moisture-proof L-α-glycerylphosphorylcholine comprises the following components in percentage by weight:

| | |
|---|---|
| L-α-glycerylphosphorylcholine | 71% |
| silicon dioxide | 1% |
| maltodextrin | 20% |
| hydroxypropyl methylcellulose | 8%. |

The preparation method was as follows: hydroxypropyl methylcellulose of a prescribed amount is prepared into an ethanol solution with a solid content of 5%. It was then sprayed on L-α-glycerylphosphorylcholine, maltodextrin and silicon dioxide of prescribed amounts. After granulation, it was sieved with a 40-mesh sieve to obtain the moisture-proof L-α-glycerylphosphorylcholine.

### Example 3 Preparation of Moisture-Proof L-α-Glycerylphosphorylcholine

A moisture-proof L-α-glycerylphosphorylcholine comprises the following components in percentage by weight:

| | |
|---|---|
| L-α-glycerylphosphorylcholine | 91% |
| silicon dioxide | 1% |
| maltodextrin | 6% |
| hydroxypropyl methylcellulose | 2%. |

The preparation method was as follows: hydroxypropyl methylcellulose of a prescribed amount is prepared into an ethanol solution with a solid content of 5%. It was then sprayed on L-α-glycerylphosphorylcholine, maltodextrin and silicon dioxide of prescribed amounts. After granulation, it was sieved with a 40-mesh sieve to obtain the moisture-proof L-α-glycerylphosphorylcholine.

### Example 4 Preparation of Moisture-Proof L-α-Glycerylphosphorylcholine

A moisture-proof L-α-glycerylphosphorylcholine comprises the following components in percentage by weight:

| | |
|---|---|
| L-α-glycerylphosphorylcholine | 91% |
| silicon dioxide | 1% |
| mannitol | 5% |
| hydroxypropyl methylcellulose | 2% |
| lecithin | 1%. |

The preparation method was as follows: hydroxypropyl methylcellulose of a prescribed amount and lecithin of a prescribed amount were each prepared into an ethanol solution with a solid content of 5%. The above ethanol solutions were then successively sprayed on L-α-glycerylphosphorylcholine, mannitol and silicon dioxide of prescribed amounts. After granulation, it was sieved with a 40-mesh sieve to obtain the moisture-proof L-α-glycerylphosphorylcholine.

### Example 5 Preparation of Moisture-Proof L-α-Glycerylphosphorylcholine

A moisture-proof L-α-glycerylphosphorylcholine comprises the following components in percentage by weight:

| | |
|---|---|
| L-α-glycerylphosphorylcholine | 91% |
| silicon dioxide | 1% |
| hydroxypropyl methylcellulose | 2% |
| anhydrous sodium citrate | 6%. |

The preparation method was as follows: hydroxypropyl methylcellulose of a prescribed amount and anhydrous sodium citrate of a prescribed amount were each prepared into an ethanol solution with a solid content of 5%. The above ethanol solutions were then successively sprayed on L-α-glycerylphosphorylcholine and silicon dioxide of prescribed amounts. After granulation, it was sieved with a 40-mesh sieve to obtain the moisture-proof L-α-glycerylphosphorylcholine.

### Example 6 Preparation of Moisture-Proof L-α-Glycerylphosphorylcholine

A moisture-proof L-α-glycerylphosphorylcholine comprises the following components in percentage by weight:

| | |
|---|---|
| L-α-glycerylphosphorylcholine | 90% |
| microcrystalline cellulose | 5% |
| hydroxypropyl methylcellulose | 4% |
| silicon dioxide | 1%. |

The preparation method was as follows: respectively sieving L-α-glycerylphosphorylcholine, microcrystalline cellulose, hydroxypropyl methylcellulose and silicon dioxide, and uniformly mixing according to the above formula. After mixing uniformly, dry granulation was carried out, and the intermediate moisture-proof L-α-glycerylphosphorylcholine was prepared at a feeding speed of 5-10 rpm, a roller pressure of 5 MPa, a tabletting pressure of 5-25 MPa and a granulating speed of 10 rpm. It was then sieved with a 20-80 mesh sieve to obtain the moisture-proof L-α-glycerylphosphorylcholine.

### Example 7 Preparation of Moisture-Proof L-α-Glycerylphosphorylcholine

A moisture-proof L-α-glycerylphosphorylcholine comprises the following components in percentage by weight:

| | |
|---|---|
| L-α-glycerylphosphorylcholine | 90% |
| microcrystalline cellulose | 8% |
| hydroxypropyl methylcellulose | 1% |
| silicon dioxide | 1%. |

The preparation method was as follows: respectively sieving L-α-glycerylphosphorylcholine, microcrystalline cellulose, hydroxypropyl methylcellulose and silicon dioxide, and uniformly mixing according to the above formula. After mixing uniformly, dry granulation was carried out, and the intermediate moisture-proof L-α-glycerylphosphorylcholine was prepared at a feeding speed of 5-10 rpm, a roller pressure of 5 MPa, a tabletting pressure of 5-25 MPa and a granulating speed of 10 rpm. It was then sieved with a 20-80 mesh sieve to obtain the moisture-proof L-α-glycerylphosphorylcholine.

### Example 8 Preparation of Moisture-Proof L-α-Glycerylphosphorylcholine

A moisture-proof L-α-glycerylphosphorylcholine comprises the following components in percentage by weight:

| | |
|---|---|
| L-α-glycerylphosphorylcholine | 80% |
| microcrystalline cellulose | 15% |
| hydroxypropyl methylcellulose | 4% |
| silicon dioxide | 1%. |

The preparation method was as follows: respectively sieving L-α-glycerylphosphorylcholine, microcrystalline cellulose, hydroxypropyl methylcellulose and silicon dioxide, and uniformly mixing according to the above formula. After mixing uniformly, dry granulation was carried out, and the intermediate moisture-proof L-α-glycerylphosphorylcholine was prepared at a feeding speed of 5-10 rpm, a roller pressure of 5 MPa, a tabletting pressure of 5-25 MPa and a granulating speed of 10 rpm. It was then sieved with a 20-80 mesh sieve to obtain the moisture-proof L-α-glycerylphosphorylcholine.

### Example 9 Preparation of Moisture-Proof L-α-Glycerylphosphorylcholine

A moisture-proof L-α-glycerylphosphorylcholine comprises the following components in percentage by weight:

| | |
|---|---|
| L-α-glycerylphosphorylcholine | 80% |
| microcrystalline cellulose | 10% |
| hydroxypropyl methylcellulose | 9% |
| silicon dioxide | 1%. |

The preparation method was as follows: respectively sieving L-α-glycerylphosphorylcholine, microcrystalline cellulose, hydroxypropyl methylcellulose and silicon dioxide, and uniformly mixing according to the above formula. After mixing uniformly, dry granulation was carried out, and the intermediate moisture-proof L-α-glycerylphosphorylcholine was prepared at a feeding speed of 5-10 rpm, a roller pressure of 5 MPa, a tabletting pressure of 5-25 MPa and a granulating speed of 10 rpm. It was then sieved with a 20-80 mesh sieve to obtain the moisture-proof L-α-glycerylphosphorylcholine.

### Example 10 Preparation of Moisture-Proof L-α-Glycerylphosphorylcholine

A moisture-proof L-α-glycerylphosphorylcholine comprises the following components in percentage by weight:

| | |
|---|---|
| L-α-glycerylphosphorylcholine | 50% |
| microcrystalline cellulose | 40% |
| hydroxypropyl methylcellulose | 8% |
| silicon dioxide | 2%. |

The preparation method was as follows: respectively sieving L-α-glycerylphosphorylcholine, microcrystalline cellulose, hydroxypropyl methylcellulose and silicon dioxide, and uniformly mixing according to the above formula. After mixing uniformly, dry granulation was carried out, and the intermediate moisture-proof L-α-glycerylphosphorylcholine was prepared at a feeding speed of 5-10 rpm, a roller pressure of 5 MPa, a tabletting pressure of 5-25 MPa and a granulating speed of 10 rpm. It was then sieved with a 20-80 mesh sieve to obtain the moisture-proof L-α-glycerylphosphorylcholine.

### Example 11 Preparation of Moisture-Proof L-α-Glycerylphosphorylcholine

A moisture-proof L-α-glycerylphosphorylcholine comprises the following components in percentage by weight:

| | |
|---|---|
| L-α-glycerylphosphorylcholine | 50% |
| microcrystalline cellulose | 40% |
| hydroxypropyl methylcellulose | 5% |
| silicon dioxide | 5%. |

The preparation method was as follows: respectively sieving L-α-glycerylphosphorylcholine, microcrystalline cellulose, hydroxypropyl methylcellulose and silicon dioxide, and uniformly mixing according to the above formula. After mixing uniformly, dry granulation was carried out, and the intermediate moisture-proof L-α-glycerylphosphorylcholine was prepared at a feeding speed of 5-10 rpm, a roller pressure of 5 MPa, a tabletting pressure of 5-25 MPa and a granulating speed of 10 rpm. It was then sieved with a 20-80 mesh sieve to obtain the moisture-proof L-α-glycerylphosphorylcholine.

### Example 12 Investigation on Hygroscopicity of Moisture-Proof L-α-Glycerylphosphorylcholine

Specific test method: A dry glass weighing bottle with plug (outer diameter 50 mm, height 15 mm) was placed in an artificial climate box (temperature 25° C., relative humidity 75%) one day before the test, and was accurately weighed (m₁). An appropriate amount of test sample was spread in the weighing bottle (thickness of test sample is generally about 1 mm), and it was accurately weighed (m₂). The weighing bottle was opened and placed with the bottle cap under the above-mentioned constant temperature and humidity conditions. The weighing bottle was taken out at 5 min, 10 min, 20 min, 30 min, 60 min, 120 min and 180 min respectively, and accurately weighed (m₃). The weight gain percentage was calculated according to the calculation formula: weight gain percentage=100%× (m₃−m₂)/(m₂−m₁). The hygroscopicity and weight gain results of moisture-proof L-α-glycerylphosphorylcholine samples are shown in Table 1.

TABLE 1

| Sample | Content | 5 min | 10 min | 20 min | 30 min | 60 min | 120 min | 180 min |
|---|---|---|---|---|---|---|---|---|
| Raw material 1 | 100% | 3.0% | 4.2% | 7.4% | 9.5% | 12.3% | 14.6% | 18.5% |
| Raw material 2 | | 3.2% | 4.1% | 7.2% | 9.4% | 12.6% | 14.4% | 18.7% |
| Commercial | | 0.7% | 1.7% | 2.3% | 3.3% | 4.7% | 6.6% | 7.4% |
| sample 1 | | | | | | | | |
| Commercial | | 3.0% | 4.1% | 6.9% | 8.9% | 12.4% | 14.4% | 18.0% |
| product | | | | | | | | |
| Example 1 | 51% | 0.3% | 0.3% | 0.4% | 0.6% | 1.2% | 1.5% | 1.9% |
| Example 2 | 71% | 0.4% | 0.9% | 1.0% | 1.5% | 1.9% | 2.6% | 3.8% |
| Example 3 | 91% | 0.6% | 1.0% | 1.8% | 3.1% | 4.2% | 5.0% | 5.4% |
| Example 4 | 91% | 0.6% | 1.1% | 1.9% | 2.8% | 4.1% | 4.7% | 5.7% |
| Example 8 | 80% | 0.6% | 1.4% | 2.0% | 3.4% | 4.1% | 4.9% | 6.3% |
| Example 9 | 80% | 0.5% | 1.2% | 2.1% | 3.2% | 4.0% | 4.6% | 6.0% |
| Example 10 | 50% | 0.2% | 0.8% | 1.3% | 1.9% | 2.3% | 3.1% | 4.0% |
| Example 11 | 50% | 0.2% | 0.6% | 1.1% | 2.0% | 2.5% | 3.0% | 4.1% |

As can be seen from Table 1, the moisture-proof L-α-glycerylphosphorylcholine prepared by the invention each has obvious moisture-proof and deliquescence-resistant effects compared with raw materials and commercial products under high humidity conditions. And with the increase of the amounts of auxiliary materials, the moisture-proof effect is more obvious. When the moisture-proof L-α-glycerylphosphorylcholine of the invention comprises, by weight, not less than 51%, 52%, 53%, 54%, 55%, 56%, 57%, 58%, 59%, 60%, 61%, 62%, 63%, 64%, 65%, 70%, 75%, 80%, 85%, 90%, 95%, 97%, 99% of L-α-glycerylphosphorylcholine, it can achieve more obvious moisture-proof, moisture absorption resistant and deliquescence resistant effects than raw materials and commercial products under high humidity conditions. The moisture absorption weight gain of the moisture-proof L-α-glycerylphosphorylcholine of the invention is not more than 5.0%, 5.5%, 6.0%, or 6.5% after being placed under high humidity conditions, specifically at temperature of 25° C. and relative humidity of 75% for 120 min; not more than 6.3%, 6.5%, 6.8%, or 7.0%, after being placed for 180 min; not more than 4.2%, 4.5% after being placed for 60 min.

### Example 17 Comparative Study on Fluidity, Adhesiveness and Deliquescence of Moisture-Proof L-α-Glycerylphosphorylcholine in Artificial Climate Box (Temperature 25° C., Relative Humidity 75%)

Study method: 5 g of sample was weighed into a weighing dish (60*30 mm), and placed in an open position at 25° C./75% RH to observe the moisture-proof effect for 0˜3 h. The fluidity results of moisture-proof L-α-glycerylphosphorylcholine of some examples are shown in Table 2.

TABLE 2

| | | | Repose | | Repose |
|---|---|---|---|---|---|
| Sample | Content | Before | angle | After | angle |
| Raw | 100% | Crystalloid/particle, good | 41° | All deliquescent and | unmeasurable |
| material | | fluidity, little caking | | liquefied, without any flow | |
| Commercial | | Fine powder/fine | 36° | Severe area deliquescent anD | unmeasurable |
| sample 1 | | particle, good fluidity | | caking, without any flow | |
| Commercial | | Fine particle, good | 37° | Moderate area deliquescent | 53° |
| sample 2 | | fluidity | | and caking, poor fluidity | |
| Example 1 | 51% | Fine powder, good | 35° | Mild pseudo caking, | 40° |
| | | fluidity | | good fluidity | |
| Example 2 | 71% | Fine powder, good | 37° | Mild pseudo caking, | 41° |
| | | fluidity | | good fluidity | |
| Example 3 | 91% | Fine particle, good | 37° | Mild caking, good | 44° |
| | | fluidity | | fluidity | |
| Example 9 | 80% | Fine particle, good | 36° | Mild caking, good | 42° |
| | | fluidity | | fluidity | |

As can be seen from table 2, compared with raw materials and commercial samples, the moisture-proof L-α-glycerylphosphorylcholine prepared by the disclosed method each has high loading capacity, good moisture-proof effect and good fluidity under high humidity conditions. The repose angle of the moisture-proof L-α-glycerylphosphorylcholine is sustainably unchanged or changes not more than 8°, 10°, 12° after being placed for a period of time. The repose angle of the moisture-proof L-α-glycerylphosphorylcholine is not higher than 44°, 45°, 48° after being placed for a period of time. The repose angle of the moisture-proof L-α-glycerylphosphorylcholine is not higher than 44°, 45°, 48°, 50° after being placed at temperature of 25° C. and relative humidity of 75% for 3 h. The products prepared in Examples 3, 4 and 5 have good moisture-proof property, and at the same time, the maximum load of L-α-glycerylphosphorylcholine is realized.

See Table 3-1 for the scoring standards of some examples. See Table 3-2 for the score results of fluidity, adhesiveness and deliquescence of moisture-proof L-α-glycerylphosphorylcholine. The passing rate (%) of 20-mesh sieve of some examples was also tested, and the results are shown in Table 4.

TABLE 3-1

| | | | Caking/agglomeration |
|---|---|---|---|
| | | | (while observing wall |
| Rating/score | Fluidity | Deliquescence | adsorption) |
| Fairly good | Can flow by tilting | No deliquescence | No caking |
| (9-10) | or shaking slightly | | |
| Good (6-8) | Can flow by tilting | Deliquescence | Caking tendency or |
| | or shaking slightly | tendency or slight | little caking |
| | | deliquescence in | |
| | | small area | |
| Ordinary (5) | Can't flow by tilting, | Slight or moderate | Little caking |
| | can flow by shaking | deliquescence in a | |
| | slightly | small area | |
| Poor (2-4) | Can't flow by | Moderate area | Moderate caking |
| | shaking slightly, can | deliquescence and | |
| | flow by vibrating | liquefaction | |
| Fairly poor | can't flow by | Large area | Severe caking |
| (0-1) | vibrating, can or | deliquescence and | |
| | can't flow by | liquefaction | |
| | vibrating on table | | |

TABLE 3-2

| | 5 | 10 | 20 | 30 | 60 | 120 | 180 |
|---|---|---|---|---|---|---|---|
| Sample | min | min | min | min | min | min | min |
| Raw | 9 | 0 | 0 | 0 | 0 | 0 | 0 |
| material | | | | | | | |
| Commercial | 9 | 5 | 3 | 0 | 0 | 0 | 0 |
| product | | | | | | | |
| Example 6 | 10 | 9 | 8 | 6 | 6 | 5 | 5 |
| Example 7 | 10 | 9 | 8 | 7 | 6 | 5 | 5 |
| Example 8 | 10 | 9 | 8 | 7 | 7 | 6 | 6 |
| Example 9 | 10 | 9 | 8 | 7 | 6 | 6 | 6 |
| Example 10 | 10 | 9 | 9 | 9 | 8 | 8 | 7 |
| Example 11 | 10 | 9 | 9 | 9 | 8 | 8 | 7 |

TABLE 4

| | 5 | 10 | 20 | 30 | 60 | 120 | 180 |
|---|---|---|---|---|---|---|---|
| Sample | min | min | min | min | min | min | min |
| Raw | 97 | 35 | 24 | 2 | 0 | 0 | 0 |
| material | | | | | | | |
| Commercial | 98 | 72 | 64 | 27 | 9 | 0 | 0 |
| product | | | | | | | |
| Example 6 | 100 | 97 | 92 | 82 | 76 | 67 | 60 |
| Example 7 | 100 | 96 | 94 | 84 | 77 | 63 | 61 |
| Example 8 | 100 | 97 | 94 | 84 | 75 | 69 | 65 |
| Example 9 | 100 | 98 | 96 | 84 | 72 | 68 | 65 |
| Example 10 | 100 | 99 | 96 | 87 | 80 | 73 | 69 |
| Example 11 | 100 | 98 | 96 | 86 | 81 | 74 | 70 |

As can be seen Table 3-2, the moisture-proof L-α-glycerylphosphorylcholine prepared by the invention is at a higher level in fluidity, adhesiveness (caking) and deliquescence scores than raw materials and commercial products under high humidity conditions. With the increase of the amounts of auxiliary materials, the better the fluidity, the stronger the adhesion resistance and the better the deliquescence resistance. As can be seen from Table 4, under high humidity conditions, the passing rate of 20-mesh sieve of the moisture-proof L-α-glycerylphosphorylcholine of the invention is not less than 75%, 80%, 85%, 90%, or 92% after 20 min; not less than 50%, 65%, 70%, 75%, 80%, 82% after 30 min; not less than 50%, 60%, 65%, 70%, 72% after 60 min; not less than 50%, 60%, 63% after 120 min; and not less than 40%, 50%, 60% after 180 min. It shows that the moisture-proof L-α-glycerylphosphorylcholine of the invention has good fluidity, strong adhesion resistance and good deliquescence resistance.

Although specific embodiments and examples of the present disclosure have been illustrated herein, it will be appreciated by those skilled in the art that any modifications and variations can be made without departing from the spirit of the present disclosure. The examples and illustrations above are not intended to limit the scope of the present disclosure. Any combination of embodiments of the present disclosure, along with any obvious their extension or analogs, are within the scope of the present disclosure. Further, it is intended that the present disclosure encompass any arrangement, which is calculated to achieve that same purpose, and all such variations and modifications fall within the scope of the appended claims.

What is claimed is:

1. A moisture-proof L-α-glycerylphosphorylcholine, wherein the moisture-proof L-α-glycerylphosphorylcholine comprises, by weight, not less than 50% of L-α-glycerylphosphorylcholine.

2. The moisture-proof L-α-glycerylphosphorylcholine of claim 1, wherein the moisture-proof L-α-glycerylphosphorylcholine comprises, by weight, not less than 51%, 52%, 53%, 54%, 55%, 56%, 57%, 58%, 59%, 60% of L-α-glycerylphosphorylcholine.

3. The moisture-proof L-α-glycerylphosphorylcholine of claim 1, wherein the moisture-proof L-α-glycerylphosphorylcholine comprises, by weight, 51-99% of L-α-glycerylphosphorylcholine; 1-49% of one or more auxiliary materials selected from silicon dioxide, maltodextrin, mannitol, silica gel.

4. The moisture-proof L-α-glycerylphosphorylcholine of claim 1, wherein the moisture-proof L-α-glycerylphosphorylcholine further comprises, by weight, 1-9% of one or more auxiliary materials selected from hydroxypropyl methylcellulose, lecithin or anhydrous sodium citrate.

5. The moisture-proof L-α-glycerylphosphorylcholine of claim 1, wherein the moisture-proof L-α-glycerylphosphorylcholine comprises, by weight, 51-91% of L-α-glycerylphosphorylcholine; 1-41% of one or more of silicon dioxide, maltodextrin, mannitol; 1-8% of one or more of hydroxypropyl methylcellulose, lecithin or anhydrous sodium citrate.

6. The moisture-proof L-α-glycerylphosphorylcholine of claim 1, wherein the moisture-proof L-α-glycerylphosphorylcholine comprises, by weight, 50-90% of L-α-glycerylphosphorylcholine; 1-5% of one or more auxiliary materials selected from silicon dioxide, magnesium stearate, calcium hydrogen phosphate, calcium silicate, micro-powder silica gel; 5-40% of one or more auxiliary materials selected from microcrystalline cellulose, compressible starch, lactose or mannitol; and 1-9% of one or more auxiliary materials selected from hydroxypropyl methylcellulose, sodium alginate, Arabic gum, guar gum.

7. The moisture-proof L-α-glycerylphosphorylcholine of claim 1, wherein the moisture-proof L-α-glycerylphosphorylcholine comprises, by weight, 50-90% of L-α-glycerylphosphorylcholine; 1-5% of silicon dioxide; 5-40% of microcrystalline cellulose; 1-9% of hydroxypropyl methylcellulose.

8. The moisture-proof L-α-glycerylphosphorylcholine of claim 1, wherein the moisture-proof L-α-glycerylphosphorylcholine comprises, by weight, 50-80% of L-α-glycerylphosphorylcholine; 1-5% of silicon dioxide, 10-40% of microcrystalline cellulose, 4-9% of hydroxypropyl methylcellulose.

9. The moisture-proof L-α-glycerylphosphorylcholine of claim 1, wherein the moisture-proof L-α-glycerylphosphorylcholine is in granular or powdery form.

10. The moisture-proof L-α-glycerylphosphorylcholine of claim 9, wherein the particle size of the moisture-proof L-α-glycerylphosphorylcholine is 10-100 meshes.

11. The moisture-proof L-α-glycerylphosphorylcholine of claim 1, wherein the moisture absorption weight gain of the moisture-proof L-α-glycerylphosphorylcholine is not more than 5.0% after being placed at temperature of 25° C. and relative humidity of 75% for 120 min.

12. The moisture-proof L-α-glycerylphosphorylcholine of claim 1, wherein the moisture absorption weight gain of the moisture-proof L-α-glycerylphosphorylcholine is not more than 7.0% after being placed for a period of time.

13. The moisture-proof L-α-glycerylphosphorylcholine of claim 1, wherein the moisture absorption weight gain of the moisture-proof L-α-glycerylphosphorylcholine is not more than 7.0% after being placed at temperature of 25° C. and relative humidity of 75% for 180 min.

14. The moisture-proof L-α-glycerylphosphorylcholine of claim 1, wherein the repose angle of the moisture-proof L-α-glycerylphosphorylcholine is sustainably changes not more than 10° after being placed for a period of time.

15. The moisture-proof L-α-glycerylphosphorylcholine of claim 1, wherein the repose angle of the moisture-proof L-α-glycerylphosphorylcholine is not higher than 44° after being placed for a period of time.

16. The moisture-proof L-α-glycerylphosphorylcholine of claim 1, wherein the passing rate of 20-mesh sieve of the moisture-proof L-α-glycerylphosphorylcholine is not less than 90% after being placed for 20 min.

17. The moisture-proof L-α-glycerylphosphorylcholine of claim 1, wherein the passing rate of 20-mesh sieve of the moisture-proof L-α-glycerylphosphorylcholine is not less than 50% after being placed for 30 min.

18. The moisture-proof L-α-glycerylphosphorylcholine of claim 1, wherein the passing rate of 20-mesh sieve of the moisture-proof L-α-glycerylphosphorylcholine is not less than 50% after being placed for 180 min.

19. The moisture-proof L-α-glycerylphosphorylcholine of claim 1, wherein the moisture-proof L-α-glycerylphosphorylcholine is formulated into nutritional supplement, food, beverage or animal feed.

20. The moisture-proof L-α-glycerylphosphorylcholine of claim 1, wherein the moisture-proof L-α-glycerylphosphorylcholine is prepared into solid preparation or liquid preparation.
